(19) United States
(12) Patent Application Publication
(10) Pub. No.: US 2025/0082380 A1
(43) Pub. Date: Mar. 13, 2025

(54) BONE STABILIZATION SYSTEMS

(71) Applicant: GLOBUS MEDICAL, INC., AUDUBON, PA (US)

(72) Inventors: Gabrielle Zingalis, Philadelphia, PA (US); Peter Govey, Ardmore, PA (US); Peter Evans, Lafayette Hill, PA (US)

(21) Appl. No.: 18/956,464

(22) Filed: Nov. 22, 2024

Related U.S. Application Data
(63) Continuation of application No. 16/597,381, filed on Oct. 9, 2019, now Pat. No. 12,185,995.

(51) Int. Cl. A61B 17/80

(52) U.S. Cl. CPC A61B 17/8061; A61B 17/8057

(57) ABSTRACT

Bone plates for engaging bone members are described herein. The bone plates can include features that accommodate the underlying anatomy of different types of bone, such as the proximal portion of the ulna. The bone plate can receive one or more fasteners to secure the bone plate to the proximal ulna. A reverse angle fastener may be included to target the olecranon process of the proximal ulna.

### CROSS REFERENCE TO RELATED APPLICATIONS

This application is a continuation of U.S. patent application Ser. No. 16/597,381, filed on Oct. 9, 2019 and published as US 2021-0106368, which is hereby incorporated by reference it its entirety.

### FIELD OF THE INVENTION

The present disclosure relates to surgical devices, and more particularly, stabilization systems including plates, for example, for trauma applications.

### BACKGROUND OF THE INVENTION

The ulna is a long bone found in the forearm that extends from the elbow to the smallest finger and is found on the medial side of the forearm. It runs parallel to the radius, the other long bone in the forearm, and is the larger and longer of the two bones. The proximal ulna consists of the olecranon process posteriorly and the coronoid process anteriorly. The olecranon process and the coronoid process define the greater sigmoid notch, which articulates with the trochlea of the humerus. The lesser sigmoid notch is located on the lateral aspect of the proximal ulna and articulates with the radial head. The olecranon acts as a posterior buttress, preventing anterior translation of the ulna, and is the site of triceps attachment. Bone fracture of the proximal ulna may be caused, for example, due to falls on the forearm or outstretched arm or direct impact from an object to the forearm.

Bone fractures can be healed using plating systems. During treatment, one or more screws are placed on either side of the fracture, thereby causing compression and healing of the fracture. There is a need for improved plating systems as well as designs to optimize screw trajectories and target critical anatomical areas.

### SUMMARY OF THE INVENTION

In accordance with the application, in some embodiments, a system is provided for treating a fracture in a bone, such as a proximal ulna bone. The bone plate may have a head portion, a shaft portion, an upper surface and a lower surface configured to engage the bone. The head portion may include a first proximal-most opening, a plurality of second openings, and a third opening, with each of the openings extending from the upper surface to the lower surface. The third opening may have a protrusion on the upper surface of the plate around a first portion of the opening and a recess into the upper surface of the plate around a second portion of the opening. A first fastener is receivable within the first opening and configured to extend distally. A plurality of second fasteners are receivable within the plurality of second openings and configured to extend distally and/or laterally. A third fastener is receivable within the third opening and configured to extend in a direction reverse to the first and second fasteners. In particular, the third fastener may extend proximally towards the olecranon process of the ulna bone. The plurality of second fasteners may extend toward the coronoid process and/or the anterior cortex of the ulna.

In other embodiments, a system is provided for treating a fracture in a proximal ulna bone. The system includes a bone plate having a head portion, a shaft portion extending from the head portion, an upper surface and a lower surface configured to engage the bone. The head portion may include a first proximal-most opening generally oriented in a direction of a distal end of the ulna. The head portion may include a second plurality of openings targeted toward an anterior cortex of the ulna. The head portion may include a third plurality of openings targeted towards the anterior cortex of the ulna, and the third plurality of openings may be targeted differently than the second plurality of openings. The head portion may include a fourth plurality of openings targeted toward a coronoid process and/or laterally toward a lateral cortex of the ulna. The head portion may include a fifth opening targeted towards an olecranon process of the ulna bone. A first fastener is receivable within the first opening and configured to extend distally. A plurality of second fasteners are receivable within the plurality of second openings and configured to extend distally, anteriorly, and/or laterally. A plurality of third fasteners are receivable within the plurality of third openings and configured to extend distally, anteriorly, and/or laterally. A plurality of fourth fasteners are receivable within the plurality of fourth openings and configured to extend distally, anteriorly and/or laterally. A fifth fastener is receivable within the fifth opening and configured to extend in a direction reverse to all of the first, second, third, and fourth fasteners. The fifth opening may have a protrusion on the upper surface of the plate around a first portion of the opening and a recess into the upper surface of the plate around a second portion of the opening. The fifth fastener may extend proximally towards the olecranon process of the ulna bone.

In other embodiments, a bone plate is provided for treating a fracture in a proximal ulna bone. The bone plate includes a head portion, a shaft portion extending from the head portion, an upper surface and a lower surface configured to engage the bone. The head portion may include a first proximal-most opening generally oriented in a first direction of a distal end of the ulna. The head portion may include a second plurality of openings nominally targeted toward an anterior cortex of the ulna. The head portion may include a third plurality of openings targeted towards the anterior cortex, and the third plurality of openings may be targeted differently than the second plurality of openings. The head portion may include a fourth plurality of openings targeted toward a coronoid process and/or laterally toward a lateral cortex of the ulna. The head portion may include a fifth opening targeted towards the olecranon process of the ulna bone. The bone plate may be a universal plate where the shaft portion is straight in-plane or a handed plate where the shaft portion is curved with a left-handed or a right-handed curvature, thereby accommodating an ulnar bow.

In yet other embodiments, a method of securing a bone plate to an ulna bone includes positioning the bone plate against a proximal portion of the ulna bone, wherein the head portion is configured to contact the olecranon process and the shaft portion is configured to contact the shaft of the ulna bone. A fastener is secured through an elongate opening, thereby allowing for provisional placement of the plate and proximal-distal and/or medial-lateral adjustment of plate. After proper anatomical placement is achieved, additional fasteners may be secured through the head portion and/or the shaft portion of the plate. For example, a first plurality of fasteners may be positioned through polyaxial openings along the shaft; and a second plurality of fasteners may be positioned through openings in the head portion and targeted towards the distal end of the ulna, the coronoid process, the lateral cortex, the anterior cortex, and/or the olecranon process. In one embodiment, the plate is configured to target at least six proximal points of fixation with medial-lateral splay built in to resist the triceps and reduce pullout.

Also provided are kits including plates of varying shapes and sizes, fasteners including locking and non-locking screws, bone anchors, k-wires, insertion tools, sutures, suture buttons, and components for installing the same.

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIG. 1 is a view of a bone plate positioned against and secured to bone in accordance with some embodiments of the present application.

FIG. 2 is a top view of a left sided low-profile olecranon plate in accordance with one embodiment of the present application.

FIG. 3 is a top view of a universal olecranon plate in accordance with one embodiment of the present application.

FIG. 4 is a side view of the proximal segment of an olecranon plate positioned against and secured to bone in accordance with one embodiment of the present application.

FIG. 5 is a bottom view of the proximal segment of the olecranon plate shown in FIG. 4.

FIG. 6 is a top view of the proximal segment of the olecranon plate shown in FIG. 4.

FIG. 7 is a side perspective view of the proximal segment of another embodiment of an olecranon plate with the bone fasteners having overlapping trajectories.

FIG. 8 is a close-up top view of overlapping hole identifiers for the proximal plate segment according to one embodiment.

FIG. 9 is a side perspective view of a plate with a reverse angle fastener according to one embodiment.

FIG. 10 is a close-up view of the head of a reverse angle fastener and an eyebrow or protrusion on the top surface of the plate according to one embodiment.

FIG. 11 is a side perspective view of the proximal segment of the olecranon plate according to one embodiment.

FIG. 12 is a close-up top view of a fastener positioned in a two-axis positioning slot according to one embodiment.

FIG. 13 is a close-up top view of the two-axis positioning slot shown in FIG. 12.

FIG. 14 is a close-up side perspective view of an olecranon plate with suture holes according to one embodiment.

FIG. 15 is a close-up top view of the plate with suture holes shown in FIG. 14.

FIG. 16 is a close-up top view of a plate with a central suture hole design in accordance with one embodiment.

FIG. 17 is a close-up top view of a suture button recess according to one embodiment.

FIG. 18 is a top perspective view of one embodiment of the olecranon plate having a plurality of scallops along one or more sides of the plate.

FIG. 19 is a close-up perspective view of a distal segment of one embodiment of the olecranon plate having a concave bottom surface.

FIG. 20 is a close-up cross-sectional view of a polyaxial opening having a locking fastener therein.

FIG. 21 is a close-up cross-sectional view of the polyaxial opening shown in FIG. 20 having a non-locking fastener therein.

FIG. 22 is a close-up cross-sectional view of an undercut stacked hole geometry, which may be included in one or more embodiments of the plate.

### DETAILED DESCRIPTION OF THE INVENTION

Embodiments of the present application are generally directed to devices, systems and methods for bone stabilization. Some embodiments are directed to bone plates that extend across bone members to treat one or more fractures. In one exemplary embodiment, one or more plates may be suitable for the fixation of fractures and fragments of the proximal ulna, for example.

Although plates are described herein for the treatment of the proximal ulna, it will be appreciated that the plates may be adapted to contact one or more of a radius, a femur, a distal tibia, a proximal tibia, a proximal humerus, a distal humerus, a clavicle, a fibula, bones of the foot, bones of the hand, or other suitable bone or bones. The bone plates may be curved, contoured, straight, or flat. The plates may have a head portion that is contoured to match a particular bone surface, such as an olecranon process. In addition, the plates may have a shaft portion that is contoured to match a bone surface, such as the shaft of the ulna. The plates may be adapted to secure small or large bone fragments, single or multiple bone fragments, or otherwise secure one or more fractures. In particular, the systems may include a series of trauma plates and screws designed for the fixation of fractures and fragments in the proximal portion of the ulna. Different bone plates may be used to treat various types and locations of fractures.

In some embodiments, one or more of the plates described herein can comprise olecranon plates. These plates can be used by a surgeon as an internal fixation device for a variety of fracture patterns of the proximal ulna. The one or more plates can provide a number of advantages, as will be discussed further below. In particular, the plates are designed to better accommodate anatomical features, such as the olecranon process, the ulnar bow, etc. Although fixation of the proximal ulna is exemplified herein, it will be appreciated by one skilled in the art that one or more features of the plates may be suitable in other anatomical locations.

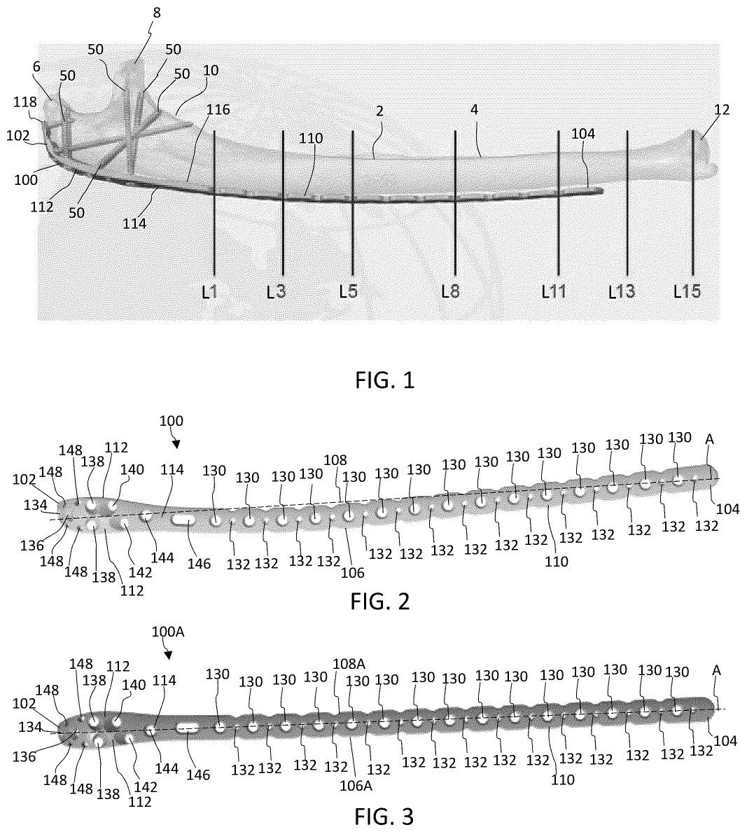

FIG. 1 shows a bone plate 100 affixed to bone in accordance with one embodiment of the present application. The bone plate 100 comprises an olecranon plate that is configured to be attached to a proximal portion of an ulna bone 2. The olecranon plate 100 comprises a proximal portion 102 and a distal portion 104. The proximal portion 102 comprises a head portion 112 that transitions into a shaft portion 110. The plate 100 includes an upper surface 114 and an opposed lower surface 116 configured to contact the bone 2. The shaft portion 110 of the bone plate 100 is configured such that the lower surface 116 resides along the shaft 4 of the ulna 2, and the lower surface 116 of the head portion 112 and/or proximal portion 102 of the bone plate 100 is configured to rest against the olecranon process 6 of the proximal end of the ulna 2. The olecranon process 6 forms the prominence of the elbow.

The plate 110 includes a first sidewall 106 and a second sidewall 108, opposite to the first sidewall 106, extending along its length between the proximal and distal portions 102, 104. Along the length of the bone plate 100 are one or more holes or openings 130, 134, 136, 138, 140, 142, 144, 146, 180 for receiving fasteners, screws, anchors, or the like therein. As shown in FIG. 1, the one or more plates 100 may be represented in a series of lengths. The lengths may be based on the number of holes or openings 130 provided in the shaft portion 110 of the plate 100 (namely, L1 is 1-hole, L3 is 3-holes, L5 is 5-holes, L8 is 8-holes, L11 is 11-hole, L13 is 13-holes, and L15 is 15-holes). Thus, different length plates 100 may be selected based on the anatomy of the patient, the type, location and number of fractures, or surgeon preference. Although these lengths including the specified number of holes 130 within the shaft portion 110 are exemplified, it is envisioned that other suitable numbers and configurations of holes or openings may be provided.

FIG. 2 depicts a low-profile plate 100 with an L15 length (e.g., having fifteen screw openings 130 along the shaft portion 110 of the plate 100). The low-profile plate 100 shown in FIG. 2 is left-sided and is configured to accommodate the anatomy of an ulnar bow. In other words, the plate 100 is curved along its length relative to a central longitudinal axis A extending between its proximal and distal ends 102, 104. With the left-sided curvature, the first side 106 is generally convex and the second side 108 is generally concave. It will be appreciated that a right-sided configuration (not shown) may also be available, which would be reversed, or a mirror image of the plate shown.

In an alternative embodiment shown in FIG. 3, the low-profile plate 100A does not have specific left and right-sided designs. Instead, the low-profile plate 100A is a universal option, which has a straight shaft in-plane. In other words, the plate 100A extends along the central longitudinal axis A such that there is no curvature of the first and second sides 106A, 108A. Otherwise, the features of the plate 100A are the same or similar to the features of plate 100 and will be identified with the same reference numerals.

The bone plate 100, 100A includes one or more openings configured to receive one or more fasteners 50. The fasteners may include screws (e.g., screws 50A, 50B), anchors, pins, bolts, or other suitable means for fixation of the plate to bone. The fasteners may be locking, non-locking, threaded, partially threaded, curved, expandable, or otherwise configured to anchor the plate to bone. In some embodiments, one or more of the plates 100, 100A include locking and/or non-locking holes. Locking holes and locking fasteners may be useful for patients that have weaker bone. In addition, these may be helpful to prevent screw backout. Non-locking holes and fasteners may be useful for patients that have strong bone.

The bone plates described herein can include a combination of locking holes and non-locking holes, only locking holes, or only non-locking holes. Locking holes comprise one or more openings that accept one or more locking fasteners. The one or more openings can be partially or fully threaded, thread-forming, or otherwise configured to allow locking attachment of the fastener to the hole. In some embodiments, the holes comprise stacked or polyaxial locking holes, which can accept both locking and non-locking fasteners. In some embodiments, the locking fasteners include heads that are at least partially threaded. The locking fasteners can be monoaxial or polyaxial.

Non-locking holes comprise one or more openings for accepting one or more non-locking fasteners. The one or more openings are at least in part non-threaded. In some embodiments, these openings include non-threaded or stacked openings, which can accept both locking and non-locking fasteners. In some embodiments, the holes comprise stacked or polyaxial locking holes, which can accept both locking and non-locking fasteners. The non-locking fasteners can be monoaxial or polyaxial.

FIGS. 20 and 21 show examples of polyaxial screw hole geometry, which enables the use of locking fasteners 50A (FIG. 20) and non-locking fasteners 50B (FIG. 21). The fasteners 50A, 50B include a head portion 52A, 52B and a shaft portion 54A, 54B configured to engage bone, respectively. In FIG. 20, the locking mechanism is such that the screw head 52A has self-forming threads that work by displacement of the plate material. The screw 50A may be inserted within an angular cone of 40° inclusive where the force required to dislodge the head 52A of the screw at maximum angulation is sufficient to maintain angular stability, when compared to screws inserted perpendicular to the plate. Any of the holes or openings in the plate 100, 100A may be shaped such that the fastener 50A, 50B may be inserted at different angles. The geometry of the opening is conducive to catching the threads on the head portion 52A of the fastener 50A and to reduce the axial force necessary to initiate the thread formation. In FIG. 21, the head portion 52B is non-locking and does not include any threads.

An upper portion of the hole may be tapered 28, for example, with a conical straight tapered surface cut through the top surface 114 of the plate 100 for clearance of the head portion 52A, 52B of the fastener 50A, 50B during off angle insertion. A lower portion of hole may further be tapered 29, for example, with a conical straight tapered surface cut through the bottom surface 116 of the plate 100, 100A for clearance of the shaft portion 54A, 54B during off angle insertion. The upper tapered portion 28 may be larger, for example, with a larger degree of taper than the lower tapered portion 29. The upper and/or lowered tapered portions 28, 29 may be substantially conical or may be segmented with more than one section, such as two separate conical sections having different diameters or degrees of taper.

At the intersection between the upper tapered portion 28 and the lower tapered portion 29 a narrowed central portion may have a textured portion 26. As described herein, the textured portion 26 may include threads, ridges, bumps, dimples, serrations, or other types of textured areas. The texture portion 26 may provide a positive surface for the self-forming threads to cut into, thereby helping to lock the newly formed threads into the plate 100, 100A. More detailed examples of locking and non-locking fasteners and openings are shown and described in U.S. Publication No. 2018/0049782, which is hereby incorporated by reference in its entirety for all purposes.

FIG. 22 depicts another embodiment of hole geometry that may be suitable for use with one or more embodiments of the olecranon plate 100, 100A. FIG. 22 depicts an undercut stacked hole geometry with a threaded portion 26 capable of accepting locking screws, a spherical recess 28 at the top surface 114 capable of accepting non-locking screws 50A, and an undercut 29 at the bottom surface 116 which is capable of accepting collet mechanism drill guides. More detailed examples of suitable openings are shown and described in U.S. Publication No. 2018/0049782, which is hereby incorporated by reference in its entirety for all purposes.

Turning now to the plate embodiment shown in FIG. 2, the left-sided plate 100 includes a plurality of polyaxial holes 130, 134, 136, 138, 140, 142, 144 both proximally and distally. These polyaxial holes 130, 134, 136, 138, 140, 142, 144 may have a 2.5 mm screw option, thereby enabling a thinner plate profile. For the 2.5 mm design, the thickness of the plate 100 may be held at 2 mm along the shaft 110, tapering to a thickness of less than 2.75 mm along the proximal olecranon tip to enable closing of the fascia. In the universal plate 100A shown in FIG. 3, the polyaxial holes 130, 134, 136, 138, 140, 142, 144 may have a 2.5 mm screw option, but may also feature 3.5 mm polyaxial, monoaxial, stacked or undercut stacked holes. In some embodiments, the universal plate 100A includes both 2.5 mm and 3.5 mm polyaxial openings 130, 134, 136, 138, 140, 142, 144. For the 2.5/3.5 mm universal design, the thickness of the plate 100A may be held at 3 mm along the shaft 110, tapering to a thickness of less than 2.75 mm along the proximal olecranon tip.

The plates 100, 100A may include one or more k-wire holes 132 for receiving a k-wire therein. One or more k-wires may guide the bone plate 100 to a desired surgical site. The k-wire holes 132 allow for temporary fixation of the bone plate 100, 100A to bone via the k-wire. In some embodiments, one or more of the k-wire holes 132 is unthreaded. As shown in FIGS. 2 and 3, the k-wire holes 132 may be alternatingly positioned between respective polyaxial holes 130 along the shaft portion 110 of the plate 100. In other words, a k-wire hole 132 may be positioned between every polyaxial hole 130 along the shaft 110 of the plate 100, 100A.

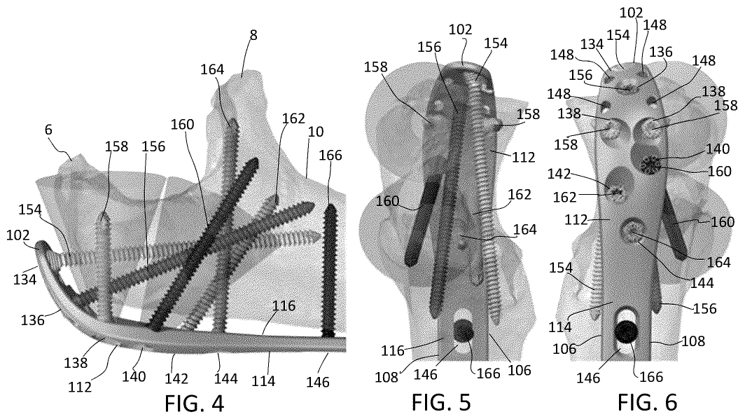

Turning now to FIGS. 4-6, the head portion 112 of the plate 100 may include one or more openings 134, 136, 138, 140, 142, 144 to aim one or more fasteners 50 along optimized screw trajectories. The trajectories of the proximal portion 102 of the plate 100 may be aligned so that their nominal axes allow for fixation in critical anatomical areas for the average proximal ulna 2.

By way of example, the head portion 112 may include one or more of the following openings 134, 136, 138, 140, 142, 144 in order to optimize fixation of the proximal ulna 2. As noted herein, the openings 134, 136, 138, 140, 142, 144 may include, for example, locking holes, non-locking holes, polyaxial holes, monoaxial holes, stacked holes, undercut stacked holes, or any combination thereof.

A first opening 134 may be provided at the proximal-most tip of the plate 100. The first opening 134 may be configured to receive a first fastener 154. The first fastener may be generally oriented such that the distal end of the fastener 154 is inserted nominally towards the distal end 12 of the ulna 2. The first fastener 154 may be positioned generally parallel to the shaft portion 110 of the plate 110. The first fastener 154 may be oriented distally, anteriorly, and/or laterally. In a preferred embodiment, the first fastener 154 is oriented distally, anteriorly, and laterally.

A second opening 136 may be provided through plate 100 such that a second fastener 156 inserted therein is nominally angled toward the anterior cortex 10 of the bone. The second opening 136 may be positioned distal to the first opening 134. The second fastener 156 may be oriented distally, anteriorly, and/or laterally. In a preferred embodiment, the second fastener 156 is oriented distally, anteriorly, and laterally. The second fastener 156 may extend laterally outward towards side 108 while first fastener 154 may extend laterally outward towards side 106.

Third and fourth openings 138 may be provided in the head portion 112 such that one or more fasteners 158 are generally positioned towards the olecranon process 6 and targeted nominally laterally outward. The fasteners 158 may be oriented anteriorly and/or laterally. In a preferred embodiment, the fasteners 158 are oriented anteriorly and laterally in diverging directions. The third fastener 158 may extend laterally outward towards side 108 while fourth fastener 158 may extend laterally outward towards side 106.

The fifth opening 140 may be provided adjacent to one of openings 138 such that fastener 160 inserted in opening 140 is generally targeted toward the coronoid process 8 and laterally toward the lateral cortex of the bone. The fastener 160 may be oriented distally, anteriorly, and/or laterally. In a preferred embodiment, the fastener 160 is oriented distally, anteriorly, and laterally.

The sixth opening 142 is configured to receive fastener 162, which may be angled toward the anterior cortex 10. The fastener 162 may be oriented distally, anteriorly, and/or laterally. In a preferred embodiment, the fastener 162 is oriented distally and anteriorly.

The seventh opening 144 is configured to receive fastener 164, which is directed towards the coronoid process 8 and may be positioned generally perpendicular to the plate 100. The fastener 164 may be oriented anteriorly and/or laterally. In a preferred embodiment, the fastener 164 is oriented only anteriorly.

The lengths of fasteners 154, 156, 158, 160, 162, 164 may be selected to target the desired anatomical areas. For example, fasteners 154, 156 may have greater lengths than fasteners 160, 164, and fasteners 158 may be shorter than fastener 162. Although certain lengths may be preferred, it will be appreciated that lengths may be selected based on patient anatomy, fracture type or location, surgeon preference, or the like.

The trajectories exemplified herein may provide for up to a total of six proximal points of fixation with medial-lateral splay built in to resist the triceps and reduce pullout. Additionally, the nominal construct enables the use of two cross-fracture screws both aimed toward a cortex and several options for rafting. Although these openings 134, 136, 138, 140, 142, 144 and fasteners 154, 156, 158, 160, 162, 164 are exemplified, it will be appreciated that other configurations may be suitable to target the proximal olecranon tip, coronoid, anterior cortex, and/or lateral cortex of the ulna 2.

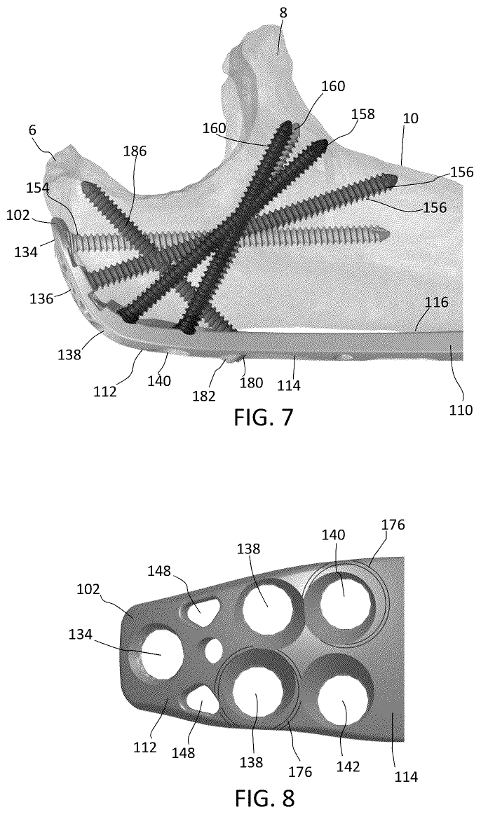

According to another embodiment, the construct may incorporate one or more overlapping screw trajectories. Proximal fixation in the olecranon 6 may be important for reducing comminuted fragments and preventing construct pullout due to triceps forces. For this reason, a surgeon may desire to have as many points of fixation proximally as possible. Overlapping screw trajectories may help to address this issue. With emphasis on FIG. 7, the overlapping trajectories may allow for more screw options to be included in the plate design, thereby giving surgeons the flexibility to pick and choose which fasteners or screws to use based on the fracture pattern and anatomy. A screw or fastener may only overlap with one or two other screws or fasteners and all overlapping holes may be denoted with one or markings 176. The marking or markings 176 may include an easily identifiable laser mark, for example, as shown in FIG. 8. The marking 176 may include a ring or partial ring around the opening 138, 140. The marking 176 may include two or more concentric rings or partial rings around one or more of the openings 138, 140.

The trajectories shown in FIG. 7 may include first opening 134, at the proximal-most tip of the head portion 112, with fastener 154 generally oriented in the direction of the distal end 12 of the ulna 2. One or more openings 136, positioning one or more fasteners 156, may be nominally targeted toward the anterior cortex 10 of the bone. One or more openings 138 with one or more fasteners 158 may be oriented towards anterior cortex 10. One or more openings 140, positioning one or more fasteners 160, may be generally targeted toward the coronoid process 8 and/or laterally toward the lateral cortex of the bone. Opening 180 with fastener 186 may be a reverse angle screw generally oriented backwards toward the olecranon process 6. In other words, fastener 186 may be positioned in a direction opposite to all of the other fasteners 154, 158, 160 in the plate 100. Fastener 154 may be positioned substantially perpendicular to the shaft portion 110 of the plate 100. Fasteners 158, 160 may be provided at an acute angle relative to the shaft portion 110 of the plate 100, and fastener 186 may be provided at an obtuse angle relative to the shaft portion 110 of the plate 100. Thus, the fasteners 154, 156, 158, 160, 186 are aimed toward different proximal targets.

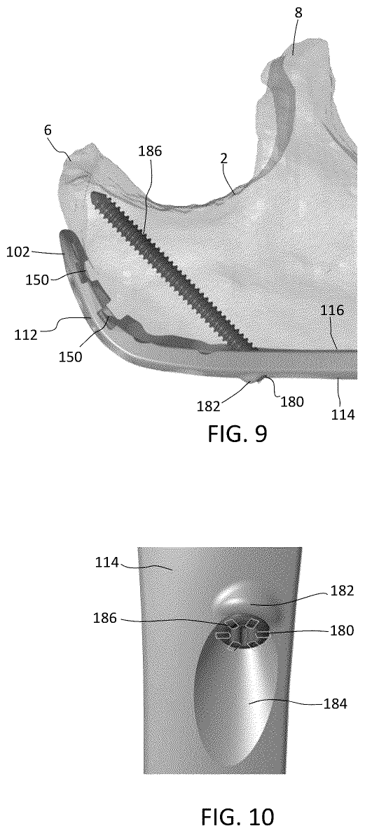

With further emphasis on FIGS. 9 and 10, the reverse angle fastener 186 may provide improved fixation at the proximal olecranon 6. The triceps is a powerful force acting on the olecranon 6 and one of the leading causes of construct pullout. To counteract this force, it may be desirable to have as many screws of fasteners crossing the fracture line as possible. Fasteners positioned in the proximal-distal direction may be considered a “home run” screw.

Another option that may be included in some embodiments of the olecranon plate 100 is a reverse angle home run screw 186. The reverse angle fastener 186 runs distal-proximal (as best seen in FIG. 7) crossing the fracture line, but in the opposite direction to all of the other fasteners in the construct. Due to its severe reverse angle, the opening 180 may include an eyebrow or protrusion 182, which may be a rounded thickness of material on the top surface 114 of the plate 100 that opposes the angle of the hole 180. The protrusion 182 may help to prevent screw thread prominence without requiring the entire plate to be thicker at that location. As best seen in FIG. 10, the opening 180 includes protrusion 182 on the upper surface 114 of the plate 100 around a first portion of the opening 180 (e.g., toward the proximal end) and a recess 184 defined into the upper surface 114 of the plate 100 around a second portion of the opening 180 (e.g., toward the distal end). The eyebrow or protrusion 182 may include a bump, ridge, or convex protrusion extending upward from the upper surface 114 of the plate 100. The protrusion 182 may be rounded, curved, angled, or otherwise configured. The protrusion 182 increases the thickness of the plate 100 at the location of the protrusion 182 and the remainder of the plate 100 maintains its low-profile thickness. Opposite to the protrusion 182 is the recess 184 cut into the upper surface 114 of the plate 100. The recess 184 may include an arcuate cutout, chamfer, or other depression. The recess 184 decreases the thickness of the plate 100 at the location of the recess 184 and the remainder of the plate 100 maintains its low-profile thickness. The recess 184 allows for insertion of the fastener 186 at the extreme angle necessary to target the olecranon process 6 of the ulna 2.

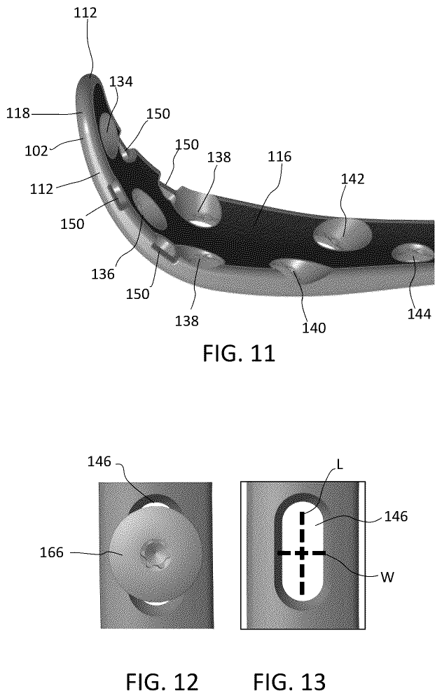

Turning now to FIG. 11, the plate 100 or a portion thereof may have an optimized plate contour. In particular, the anatomic contour along the bottom surface 116 of the plate 100 may be configured to follow the best approximation of average proximal ulna anatomy. The bottom surface 116 may be concavely curved along its length and/or its width. The plate 100 may further include a proximal impingement chamfer 118. The chamfer 118 or taper on the proximal tip of the plate 100 may help to insure minimal bony and soft-tissue impingement of the olecranon fossa and triceps.

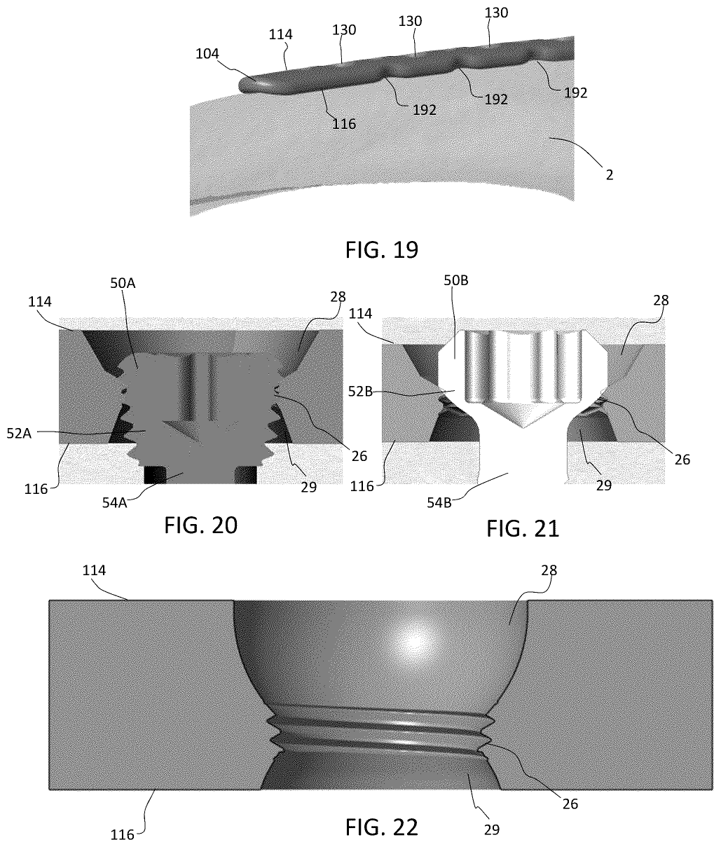

Also, with emphasis on FIG. 19, the distal portion 104 of the plate 100 may include a concavity to contour to the anatomy of the shaft 4 of the bone 2. When initially placing olecranon plates 100, the distal end 104 may kick off the bone, which may require the surgeon to reposition the plate. To reduce the likelihood of this, the shaft 110 of the plate 100 may be contoured to have a sizeable concavity that will hug the ulnar ridge and increase the resistance of kickoff.

Turning now to FIGS. 12 and 13, the plate 100, 100A may include a two-axis positioning slot 146. The slot 146 may allow for proximal-distal (P-D) adjustment and/or medial-lateral (M-L) adjustment of plate during provisional placement. The slot 146 may have a length L greater than its width W. The width W may be generally greater than an outer diameter of the shaft of a fastener 50. The length L allows for the proximal-distal adjustment of the plate 100, and the width W allows for the medial-lateral adjustment of the plate 100. The two-axis positioning slot 146 may allow surgeons to optimally center the plate position along the shaft 4 of the bone 2 prior to installing any locking screws. Additionally, the slot 146 may be etched with laser lines, for example, spaced 1 mm from each other, for more accurate adjustment. While the present embodiment illustrates a single slot 146, in some embodiments, there could be additional positioning slots.

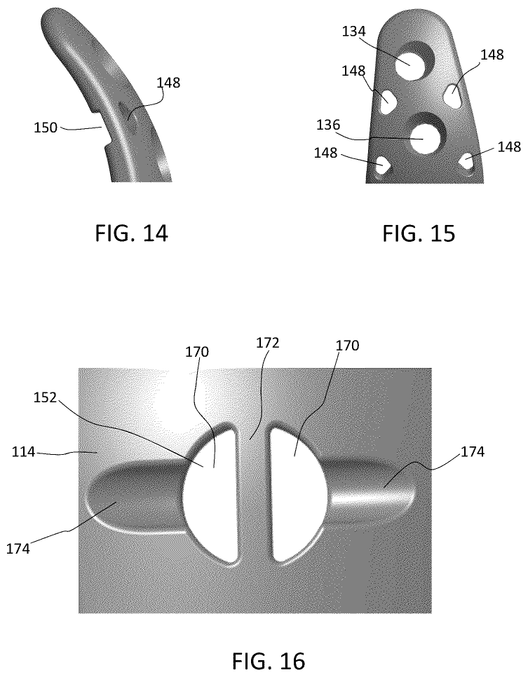

In some embodiments, the plate construct may be reinforced with augmenting sutures through the tendon, thereby increasing the ultimate load to failure following olecranon plate fixation. To accommodate this need, one or more types of suture holes 148, 152 can be incorporated into any of the plates described herein. Turning to FIGS. 14 and 15, the plate 100 may include one or more suture holes 148, which are intended for insertion of the suture along the sides of the plate 100, 100A. The suture holes 148 may be generally triangular in shape with squared or rounded undercuts 150 to case suture needle insertion. The sharp edges may be heavily broken to prevent tearing of the suture. With reference to FIG. 16, a second suture hole design 152 is intended for insertion of the suture from the top surface 114 of the plate 100 in order to avoid blockage of the needle by the bone. The suture hole 152 may be generally circular in shape with two types of undercuts to case needle insertion: a circular swept cut 174 that mimics the shape of the needle and a standard circular cut from the back surface of the plate that lifts the center tab 172 off the bone. The suture hole 152 may be divided into two sections 170 by the tab 172, thereby forming two half circle sections 170 on either side of the tab 172. The swept cuts 174 may be positioned on either side of the sections 170 and may extend generally perpendicular to the orientation of the tab 172.

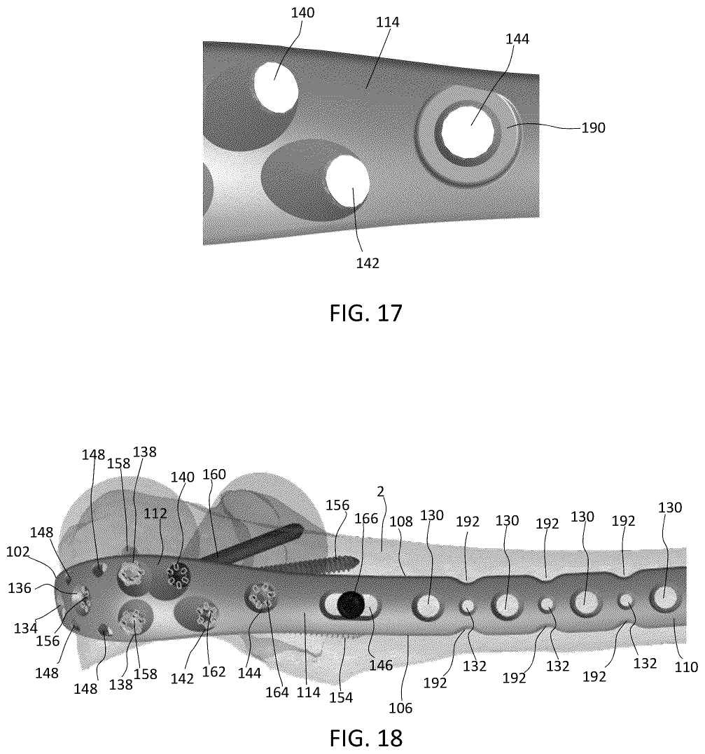

Turning now to FIG. 17, one or more of the openings may be modified for receipt of a suture button (not shown). Fractures of the coronoid are difficult to treat and, when coupled with olecranon fracture dislocation or terrible triad injuries, often require additional fixation outside of plating. To enable the fixation of the coronoid using suture in conjunction with an olecranon plate 100, a suture button recess 190 at the coronoid screw hole (e.g., opening 144) can be included in one or more embodiments of the plate. The suture button recess 190 may include a recess or indentation into the upper surface 114 of the plate 100 and surrounding the opening 144. The recess or inset 190 into the top surface 114 of the plate 100 allows for a suture button (not shown) to be firmly secured to the plate 100, thereby minimizing translational movement of the button when secured to the bone. More detailed examples of suture buttons and openings are shown and described in U.S. Publication No. 2018/0049782, which is hereby incorporated by reference in its entirety for all purposes.

Turning to FIG. 18, the edges 106, 108 of the shaft portion 110 of the plate 100 may include one or more waisted edge scallops 192. Variances in ulnar anatomy often result in surgeons in-plane bending the shaft 110 of olecranon plates during surgery. In order to make bending easier and reduce the likelihood of deformation at the shaft holes 130, one or more scallops 192 can be added between shaft holes 130. In particular, the indentations of the scallops 192 can align with each of the k-wire holes 132 provided along the shaft 110 of the plate 100. With the scallops 192 acting as a bend location guide, the surgeon can utilize plate bending irons in the system to contour the plate to best fit the anatomy of the patient.

The bone plates may be comprised of titanium, stainless steel, cobalt chrome, carbon composite, plastic or polymer-such as polyetheretherketone (PEEK), polyethylene, ultra-high molecular weight polyethylene (UHMWPE), resorbable polylactic acid (PLA), polyglycolic acid (PGA), combinations or alloys of such materials or any other appropriate material that has sufficient strength to be secured to and hold bone, while also having sufficient biocompatibility to be implanted into a body. Similarly, the bone plates may receive one or more screws or fasteners may be comprised of titanium, cobalt chrome, cobalt-chrome-molybdenum, stainless steel, tungsten carbide, combinations or alloys of such materials or other appropriate biocompatible materials. In one embodiment, the plate is constructed of a softer material (e.g., implant grade Ti, Ti alloys, and/or SS alloy) than the screw (e.g., cobalt chromium alloy, Kolsterized SS alloy, Dotized type II anodized Ti) to facilitate displacement of plate material during the thread-forming process. Although the above list of materials includes many typical materials out of which bone plates and fasteners are made, it should be understood that bone plates, fasteners, or other components may be comprised of any appropriate materials.

One skilled in the art will appreciate that the embodiments discussed above are non-limiting. While bone plates may be described as suitable for a particular location (e.g., proximal ulna) or approach, one skilled in the art will appreciate that the bone plates can be used for multiple locations and approaches. In addition, while bone plates are described as having particular holes (e.g., locking or non-locking), one skilled in the art will appreciate that any of the bone plates can include locking, non-locking or a combination of locking and non-locking holes. In addition to the bone plates, fasteners, and instruments described above, one skilled in the art will appreciate that these described features can be used with a number of trauma treatment instruments and implants, including external fixators, ring fixators, rods, and other plates and screws. It will also be appreciated that one or more features of one embodiment may be partially or fully incorporated into one or more other embodiments described herein.

1. An olecranon plate comprising:
a curved head portion configured to conform to an olecranon;
a shaft portion extending from the curved head portion and configured to extend towards a distal end of an ulna;
an upper surface comprising the curved head portion and the shaft portion;
a lower surface comprising the curved head portion and the shaft portion and configured to contact the ulna;
a first sidewall extending between the upper surface and the lower surface along a first side of the olecranon plate; and
a second sidewall extending between the upper surface and the lower surface along a second side of the olecranon plate,
wherein the shaft portion is curved with a left-handed or a right-handed curvature and configured for a left ulna or a right ulna, respectfully.

2. The olecranon plate of claim 1, wherein the olecranon plate is left-handed and the first sidewall of the shaft portion is convex and the second sidewall of the shaft portion is concave.

3. The olecranon plate of claim 1, wherein the olecranon plate is right-handed and the first sidewall of the shaft portion is concave and the second sidewall of the shaft portion is convex.

4. The olecranon plate of claim 1, comprising a first opening in the head portion configured to received a first fastener, wherein the first opening is configured to receive the first fastener such that the first fastener extends toward a distal end of the ulna.

5. The olecranon plate of claim 4, comprising a plurality of second openings positioned distally of the first opening, wherein each second opening of the plurality of second openings is configured to receive a second fastener, wherein each second opening is configured to receive a second fastener such that each second fastener extends towards one or a coronoid process of the ulna or an anterior cortex of the ulna.

6. The olecranon plate of claim 5, comprising a third opening positioned distally of the plurality of second openings, wherein the third opening is configured to receive a third fastener, wherein the third opening is configured to receive the third fastener such that the third fastener extends towards an olecranon process of the ulna.

7. The olecranon plate of claim 1, comprising a two-axis positioning slot positioned on the shaft portion configured to receive a positioning fastener, wherein the two-axis positioning slot is configured to allow the positioning fastener to adjust the olecranon plate in both a proximal-distal direction and a medial-lateral direction.

8. The olecranon plate of claim 1, wherein a profile thickness of the olecranon plate is 2 mm along the shaft and tapers to less than 2.75 mm along the proximal end of the head portion to enable closing of the fascia.

9. The olecranon plate of claim 1, wherein the first sidewall and the second sidewall define waisted edge scallops.

10. The olecranon plate of claim 9, wherein the wasted edge scallops of the first sidewall define a first indentation and the waisted edge scallops of the second sidewall define a second indentation, and the first indentation and the second indentation are aligned with a k-wire hole.

11. A system for treating a fracture in a proximal ulna bone, the system comprising:
an olecranon plate comprising:
a curved head portion configured to conform to an olecranon;
a shaft portion extending from the curved head portion and configured to extend towards a distal end of an ulna;
an upper surface comprising the curved head portion and the shaft portion;
a lower surface comprising the curved head portion and the shaft portion and configured to contact the ulna;
a first sidewall extending between the upper surface and the lower surface along a first side of the olecranon plate;
a second sidewall extending between the upper surface and the lower surface along a second side of the olecranon plate,
wherein the shaft portion is curved with a left-handed or a right-handed curvature and configured for a left ulna or a right ulna, respectfully; and
a plurality of fasteners configured to fasten the olecranon plate to the ulna.

12. The system of claim 11, wherein the olecranon plate is left-handed and the first sidewall of the shaft portion is convex and the second sidewall of the shaft portion is concave.

13. The system of claim 11, wherein the olecranon plate is right-handed and the first sidewall of the shaft portion is concave and the second sidewall of the shaft portion is convex.

14. The system of claim 11, wherein the olecranon plate comprises a first opening in the head portion configured to received a first fastener of the plurality of fasteners, wherein the first opening is configured to receive the first fastener such that the first fastener extends toward a distal end of the ulna.

15. The system of claim 14, wherein the olecranon plate comprises a plurality of second openings positioned distally of the first opening, wherein each second opening of the plurality of second openings is configured to receive a second fastener of the plurality of fasteners, wherein each second opening is configured to receive a second fastener such that each second fastener extends towards one or a coronoid process of the ulna or an anterior cortex of the ulna.

16. The system of claim 15, wherein the olecranon plate comprises a third opening positioned distally of the plurality of second openings, wherein the third opening is configured to receive a third fastener of the plurality of fasteners, wherein the third opening is configured to receive the third fastener such that the third fastener extends towards an olecranon process of the ulna.

17. The system of claim 11, wherein the olecranon plate comprises a two-axis positioning slot positioned on the shaft portion configured to receive a positioning fastener of the plurality of fasteners, wherein the two-axis positioning slot is configured to allow the positioning fastener to adjust the olecranon plate in both a proximal-distal direction and a medial-lateral direction.

18. The system of claim 11, wherein a profile thickness of the olecranon plate is 2 mm along the shaft and tapers to less than 2.75 mm along the proximal end of the head portion to enable closing of the fascia.

19. The system of claim 1, wherein the first sidewall and the second sidewall of the olecranon plate define waisted edge scallops.

20. The system of claim 19, wherein the waisted edge scallops of the first sidewall define a first indentation and the waisted edge scallops of the second sidewall define a second indentation, and the first indentation and the second indentation are aligned with a k-wire hole.
